(19) United States
(12) Patent Application Publication
(10) Pub. No.: US 2012/0165696 A1
(43) Pub. Date: Jun. 28, 2012

(54) METHOD FOR ASSESSING THE SUSCEPTIBILITY OF A HUMAN INDIVIDUAL SUFFERING FROM A PSYCHIATRIC OR NEUROLOGICAL DISORDER TO NEUROMODULATION TREATMENT

(76) Inventor: Martijn Wilco Arns, DZ Beek Ubbergen (NL)

(21) Appl. No.: 13/375,902

(22) PCT Filed: Jun. 3, 2009
(86) PCT No.: PCT/EP2009/056812

§ 371 Date: Mar. 19, 2012

(51) Int. Cl. A61B 5/0476; A61B 5/0482; A61B 5/0484

(52) U.S. Cl. 600/545; 600/544

(57) ABSTRACT

Method for assessing the susceptibility of a human individual suffering from a psychiatric or neurological disorder, in particular with depressed mood as a predominant feature, to neuromodulation treatment, in particular repetitive Transcranial Magnetic Stimulation (rTMS), the method comprising: a) Providing a dataset comprising electroencephalographic (EEG) activity and Event Related Potentials (ERP) data of said human individual; b) Assessing the susceptibility of said human individual to neuromodulation treatment based on said dataset.

The invention relates to a method for assessing the susceptibility of a human individual suffering from a psychiatric or neurological disorder to neuromodulation treatment. The invention furthermore relates to a device for use in such a method.

The treatment of psychiatric and neurological complaints is constantly subject to new insights and scientific developments. Nowadays medication and psychotherapy are applied on a large scale. Over the past few years there has also been a clear shift from a systemic—or drug treatment—to a more local treatment which is more directed to a specific area or network in the brain. These techniques are also often called Neuromodulation techniques. Examples of local neuromodulation methods are the application of repetitive Transcranial Magnetic Stimulation (rTMS or TMS), transcranial Direct Current Stimulation (tDCS), deep-brain stimulation (DBS) and EEG-biofeedback or neurofeedback. Another development, which is complementary to the above mentioned, is Personalized Medicine. Its goal is to personalize therapy on the basis of genotypic and phenotypic information in order to reach higher effectiveness for different kinds of treatments.

These new—more local—treatment options provide us with a completely new view of treatment of brain disorders whereby broad categories such as “depression” and “schizophrenia” are no longer relevant. These new treatment methods rather focus on a specific complaint (such as auditory hallucinations, depressed mood or tinnitus) rather than blindly applying the same treatment to all patients. The most critical factor of these new treatment methods is the personalizing of these treatments. The development of these new local treatment methods is therefore complimentary to the development of personalized medicine. Personalized medicine is a new approach with the goal to create more effective treatments through personalizing treatment.

Drug treatments in psychiatry only have limited effectiveness (40-60% for depression (Keller et al., 2000) and 60-80% for ADHD (Swanson et al., 1993). Considering this low to moderate effectiveness there seems to be a need for a personalized approach in treating psychiatric and neurological disorders.

It is an objective of the invention, among others, to provide a reliable, easy and/or efficient method to assess the susceptibility of a human individual to neuromodulation treatment.

In order to accomplish that objective, a method is provided for assessing the susceptibility of a human individual suffering from a psychiatric or neurological disorder, in particular with depressed mood as a predominant feature, to neuromodulation treatment, in particular repetitive Transcranial Magnetic Stimulation (rTMS), the method comprising:
a) Providing a dataset comprising electroencephalographic (EEG) activity and Event Related Potentials (ERP) data of said human individual, and;
b) Assessing the susceptibility of said human individual to neuromodulation treatment based on said dataset.

The invention is based on the recognition that brain imaging data (e.g. QEEG, ERPs, MEG, fMRI, PET scans) can be considered as a phenotype which includes both the effects of nature and nurture. Therefore it can give a reliable indication of the ‘state of the system’. This potentially makes data obtained from neuroimaging a reliable predictor for treatment outcome for treatments such as rTMS and medication (e.g. antidepressants or stimulants).

The brain function assessment according to the invention concern electroencephalographic (EEG) activity and EEG related measures (Event Related Potentials). EEG is the electrical activity from the brain and is a widely used technique to measure brain function both for research and clinical uses'. Event Related Potentials are potentials which are present in the EEG when a subject is presented with a task, where for instance auditory stimuli are presented to the subject. By averaging the EEG at exactly the presentation of these stimuli an Evoked Potential (EP) or Event Related Potential (ERP) is obtained.

Often such tasks employ different stimuli, for example in an Oddball task a frequent and infrequent tone is presented and the subject is instructed to respond to the infrequent tones, by pressing a button. The averaged EEG activity of the ‘infrequent’ tone is then the Oddball ERP and the averaged EEG activity to the ‘frequent’ tone is then the Oddball Background ERP. Where ERP is mentioned in this patent we refer to any Event Related Potential and not limited to the Oddball ERP.

In the preferred embodiment of this invention these EEG and ERP data are recorded and collected using the standardized Brain Resource Company methods and equipment. More information on these methods has also been published in the scientific literature (Gordon, 2003). To summarize, EEG data are collected from 26 scalp locations but include at least the following standard EEG locations: F3, Fz, F4, Cz and Pz. The data are at least recorded under the following conditions: a) Two minutes Eyes Open; b) Two minutes Eyes Closed and c) During an auditory oddball paradigm with an infrequent high-pitched tone (50 ms, 75 dB tone at 1000 Hz; total 280 stimuli, quasi random) and a frequent low-pitched tone (50 ms 75 dB tone at 500 Hz; total 60 stimuli, quasi random) with inter stimulus interval of 1 second. The minimum embodiment consists of a single channel of EEG and related ERP's, therefore the above does not imply multiple channels are required for this patent and single-channel recordings are explicitly also covered.

It should be noted that although the use of EEG is described, the invention also relates to the use of Magneto encephalography (MEG) data, since the MEG technique yields similar results as with EEG.

This invention applies to patients with neurological, psychological and/or psychiatric complaints in the broadest sense. In the preferred embodiment the invention applies to patients with a complaint of depressive mood (including but not limited to Major Depressive Disorder, bipolar disorder, Dysthymia, Mood Disorders or depressive mood as a comorbid psychiatric complain in for example Tinnitus or Parkinson). Below EEG and ERP predictors of favourable and unfavourable treatment response are outlined. In the broadest sense this applies to all neuromodulation treatments (rTMS, TMS, tDCS)at any scalp location, but in the preferred embodiment this specifically applies to rTMS therapy (magnetic brain stimulation or repetitive Transcranial Magnetic Stimulation) applied at any frequency to the right or left frontal cortex. The predictors for non-response implicate those people will not clinically benefit from neuromodulation treatment and the predictors for response implicate those people will respond to neuromodulation treatment.

According to a preferred embodiment, step b of assessing the susceptibility comprises:
Comparing said dataset with reference data comprising EEG and ERP data from a control group, and;
Assessing the susceptibility of said human individual to neuromodulation treatment based on said comparison.

According to this embodiment, the provided dataset is compared with reference data for assessing the susceptibility. It is however also possible to compare the provided dataset with predetermined absolute values.

According to a further preferred embodiment the susceptibility is assessed as negative in step b if the data in said dataset meets any one of the conditions chosen from the group of:
An increased P300 ERP amplitude;
Increased power of slow EEG, preferably increased power in the delta and/or theta range, and;
Low alpha peak frequency.

Tests have indicated that these conditions provide reliable indications for the susceptibility of neuromodulation treatment. As described above, the data for the various conditions can be compared to reference data. It is however also possible to establish the conditions based on absolute values of the various parameters in the conditions.

According to a further preferred embodiment said increased P300 ERP amplitude comprises a P300 Oddball amplitude. More preferably, the amplitude is considered as increased if the amplitude is higher than the average amplitude. More preferably the P300 ERP amplitude is larger than 15 μV and even more preferably larger than 20 μV. Furthermore, the dataset comprises EEG data measured at the Pz location.

According to a further preferred embodiment the increased power of slow EEG exceeds 70 μV, preferably 80 μV and more preferably 90 μV. Preferably the dataset comprises EEG data of the human individual measured with his eyes closed, preferably measured at the Pz location. It should be noted that with the delta frequency band frequencies are meant in a range of 1.5-3.5 Hz, while the theta frequency band comprises frequencies in the range of 4-7.5 Hz.

According to a further preferred embodiment the alpha peak frequency is lower than 10 Hz, preferably lower than 9 Hz and more preferably lower than 8 Hz. The alpha peak frequency comprises the maximum power in the 4-14 Hz frequency band. Preferably the data comprises EEG data of the human individual measured with his eyes closed and preferably measured at the Fz location.

According to a further preferred embodiment, the susceptibility is assessed as positive in step b if the data in said dataset meets any one of the conditions chosen from the group of:
Increased frontal fast EEG power, preferably the power in the alpha and/or beta range, and;
Increased beta EEG power.

The beta frequency band comprises the frequency range of 14.5-30 Hz.

Preferably the susceptibility is only assessed as positive in step b if none of the conditions for a negative assessment are met. The negative assessment therefore prevails over a positive assessment.

According to a further preferred embodiment the increased frontal fast EEG power is higher than 30 μV, preferably higher than 35 μV and more preferably higher than 40 μV. Preferably the data of fast EEG comprises data of the human individual measured with his eyes closed. And preferably the data of the frontal fast EEG comprises data measured from the F3, Fz and/or F4 locations.

According to a further preferred embodiment the data of the beta EEG comprises data measured from the Pz location, wherein the increased beta EEG power is higher than 20 μV, more preferably higher than 25 μV. It is also possible that the data of the beta EEG comprises data measured from the Cz location, wherein the increased beta EEG power is higher than 25 μV, more preferably higher than 30 μV. Furthermore, the data comprising beta EEG can comprise data measured from the Fz location, wherein the increased beta EEG power is higher than 15 μV, more preferably higher than 20 μV.

Preferably the data of beta EEG comprises data of the human individual measured with his eyes closed.

According to a further preferred embodiment the susceptibility is assessed as negative if the EEG data of the human individual comprises paroxysmal, neurological or epileptic EEG data.

The invention furthermore relates to a device for use in the assessment of the susceptibility of a human individual suffering from a psychiatric or neurological disorder to neuromodulation treatment according to the invention, wherein the device comprises input means for inputting a dataset comprising EEG and ERP data from the human individual and processing means for assessing the susceptibility based on said dataset and output means for outputting said assessment based on the result from the processing means.

The invention will be further elucidated using the following example and figures, wherein:

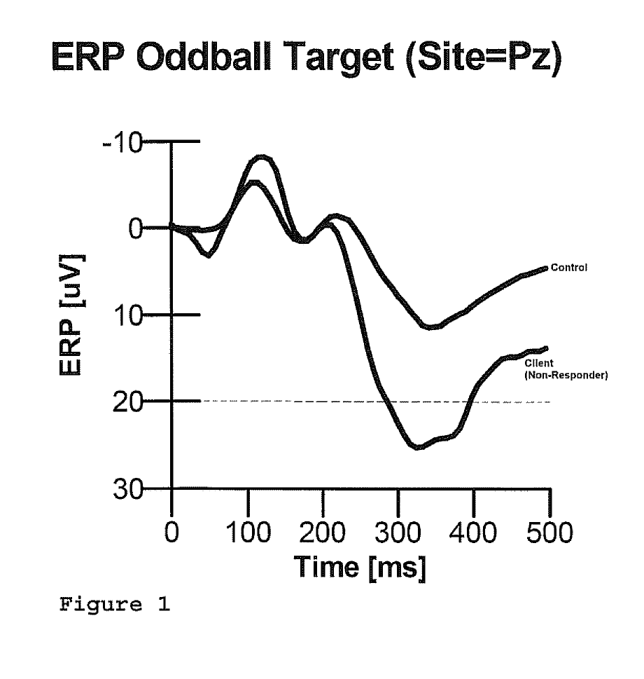

FIG. 1 shows an example of an individual ERP compared to a control group, and;

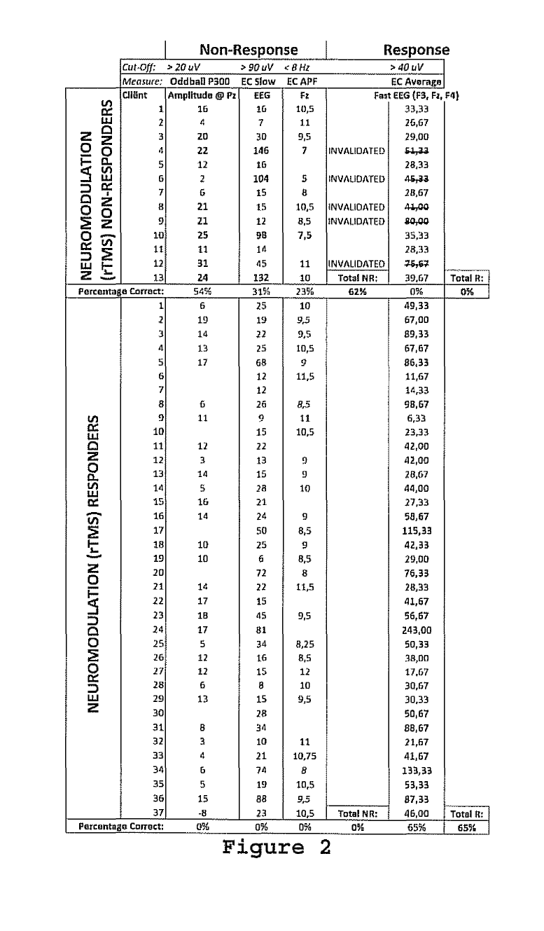

FIG. 2 shows the results of the method according to the invention.

According to the invention, the following selection method comprising steps 1-3 for assessing the susceptibility to rTMS treatment is used:

In the whole process predictors for non-response are most important and have priority over predictors for a good response. Hence, any predictor for non-response invalidates predictors for response.

Step 1) A-priori predictors for non-response are: Paroxysmal, neurological or epileptic EEG. This occurs in 3-5% of patients, even without the symptoms of epilepsy or other neurological problems. These patients need neurological follow up: This group will possibly respond to anticonvulsant medication or SMR Neurofeedback.

Step 2) Prediction of Non-response to Neuromodulation based on pre-treatment EEG and ERP parameters, any of the following observations indicates that someone will be a non-responder to neuromodulation treatment:
Increased P300 ERP amplitude (higher than average amplitude). This is a P300 oddball amplitude at Pz of more than 20 μV (microvolt), or;
Increased slow EEG (Delta and/or Theta EEG power). This is the EEG power in the Delta frequency band (1.5-3.5 Hz)+EEG power in the Theta frequency band (4-7.5 Hz) during Eyes Closed exceeding 90 μV at location Pz, or;
A slowed alpha peak frequency (the maximum power in the 4-14 Hz frequency band). This is the alpha peak frequency measured at Fz during eyes closed slower than 8 Hz.

An analysis based on 50 patients treated with rTMS showed that combining these 3 measures has resulted in a 62% correct identification of Non-Responders to rTMS treatment, with no false positive findings, also see FIG. 2.

FIG. 1 shows an example of individual data predicting a negative treatment-outcome. These and other forthcoming data will also be published after the patent has been submitted, making reference to this patent. This figure shows an example of an individual ERP (Client) compared to a control group (Control). Here we see that the P300 amplitude (the component at 300 ms.) is increased and exceeds 20 μV. This is a very clear predictor of non-response to Neuromodulation treatment.

Step 3) Prediction of Response to Neuromodulation based on pre-treatment EEG and ERP parameters, any of the observations under a. and b. means that someone will be a responder to neuromodulation treatment, without the presence of any responders as mentioned under steps 1 or 2:

a. Increased frontal fast EEG power (Alpha and/or Beta EEG Power). In the preferred embodiment this is the average EEG power in the Alpha frequency band (8-13 Hz)+EEG power in the Beta frequency band (14.5-30 Hz) during Eyes Closed at frontal locations (F3, Fz and F4) of above 40 μV, or

b. Increased beta EEG power (14.5-30 Hz). In the preferred embodiment this is a beta EEG power during eyes closed of more than 25 μV at Pz, or more than 30 μV at Cz, or more then 20 μV at Fz.

c. No presence of any of the predictors mentioned under step 1 or 2.

An analysis based on 50 patients treated with rTMS showed that in total 65% of the responders to rTMS treatment could be classified with no false positive findings. These and other forthcoming data will also be published after the patent has been submitted, making reference to this patent.

These predictors apply to all patients, and hence the presence of these EEG and ERP profiles will predict treatment outcome with high accuracy and no false positive findings are observed. These predictors might also apply in a similar or reverse order to other treatment modalities such as medication. For example it is expected that a large proportion of responders and especially non-responders to antidepressant medication shows the above EEG and ERP profiles under step 3 and hence will respond to neuromodulation treatment. Using these data patients can hence be offered the right treatment at once.

In FIG. 2 the algorithm is shown on a real dataset consisting of 50 patients treated with Neuromodulation treatment (rTMS in this case) for depressive complaints. The top 13 clients are non-responders and the bottom 37 clients are responders to this treatment.

The three measures on the left (Oddball P300 amplitude at Pz; EC Slow (Slow EEG activity during Eyes Closed at Pz) and EC APF (Alpha Peak Frequency during Eyes Closed at Fz) are the measures as described under 3: Predictors for Non-Response.

The measure on the left (EC Average Fast EEG (F3, Fz, F4) reflects the average fast EEG power from frontal sites (F3, Fz and F4) for all subjects as described under 2: Predictors for Response.

Also note the ‘INVALIDATED’ cases' demonstrating that the predictors for non-response have preference over predictors for response and hence invalidates the predictors for response.

Using these cut-off values in this example leads to correct classification of 62% of the non-responders and 65% of the responders with 0% false positives, which is important for the clinical relevance. This example demonstrates the preferred embodiment.

The present invention is not limited to the embodiment shown, but extends also to other embodiments falling within the scope of the appended claims.

1-19. (canceled)

20. A negative susceptibility predictor for a neuromodulation treatment comprising an electroencephalographic (EEG) dataset, wherein said predictor is selected from the group consisting of an increased event related potential amplitude (ERP), increased power of slow EEG, increased power in the delta range, increased power in the theta range and low alpha peak frequency as compared to a control EEG dataset.

21. The negative susceptibility predictor of claim 20, wherein said EEG dataset further comprises EEG data selected from the group consisting of paroxysmal EEG data, neurological problem EEG data, and epileptic EEG data.

22. The negative susceptibility predictor of claim 20, wherein said event related potential amplitude comprises a P300 ERP amplitude.

23. The negative susceptibility predictor of claim 22, wherein said increased P300 ERP amplitude is larger than 15 μV.

24. The negative susceptibility predictor of claim 20, wherein said increased power of delta or theta slow EEG exceeds 70 μV.

25. The negative susceptibility predictor of claim 20, wherein said low alpha peak frequency is lower than 10 Hz.

26. The negative susceptibility predictor of claim 20, wherein said EEG dataset is measured from a location selected from the group consisting of F3, Fz, F4, Cz and Pz.

27. The negative susceptibility predictor of claim 20, wherein said P300 event related potential comprises a P300 infrequent event amplitude.

28. The negative susceptibility predictor of claim 20, wherein said neuromodulation treatment is selected from the group consisting of transcranial magnetic stimulation, transcranial direct current stimulation, deep-brain stimulation, and neurofeedback.

29. The negative susceptibility predictor of claim 20, wherein said neuromodulation treatment predicts an unfavorable treatment response in a patient with a psychiatric complaint selected from the group consisting of a major depressive disorder, bipolar disorder, dysthymia, and a depressive mood disorder.

30. A positive susceptibility predictor for a neuromodulation treatment comprising an electroencephalographic (EEG) dataset, wherein said predictor is selected from the group consisting of increased alpha frontal fast EEG power, increased beta frontal fast EEG power and increased beta EEG power as compared to a control EEG dataset.

31. The positive susceptibility predictor of claim 30, wherein said increased alpha frontal fast EEG power is higher than 30 μV.

32. The positive susceptibility predictor of claim 30, wherein said increased beta frontal fast EEG power is higher than 30 μV.

33. The positive susceptibility predictor of claim 30, wherein said increased beta EEG power is higher than 20 μV when measured at a Pz location.

34. The positive susceptibility predictor of claim 30, wherein said increased beta EEG power is higher than 25 μV when measured at a Cz location.

35. The positive susceptibility predictor of claim 30, wherein said increased beta EEG power is higher than 15 μV when measured at a Fz location.

36. The positive susceptibility predictor of claim 30, wherein said neuromodulation treatment is selected from the group consisting of transcranial magnetic stimulation, transcranial direct current stimulation, deep-brain stimulation, and neurofeedback.

37. The positive susceptibility predictor of claim 30, wherein said EEG dataset is measured from a location selected from the group consisting of F3, Fz, F4, Cz and Pz.

38. The positive susceptibility predictor of claim 30, wherein said neuromodulation treatment predicts a favorable treatment response in a patient with a psychiatric complaint selected from the group consisting of a major depressive disorder, bipolar disorder, dysthymia, and a depressive mood disorder.
